(19) United States
(12) Patent Application Publication
(10) Pub. No.: US 2021/0045779 A1
(43) Pub. Date: Feb. 18, 2021

(54) INTRAVERTEBRAL SCREW

(71) Applicant: Daniel ROSENTHAL, Frankfurt am Main (DE)

(72) Inventor: Daniel ROSENTHAL, Frankfurt am Main (DE)

(21) Appl. No.: 16/964,431

(22) PCT Filed: Jan. 23, 2019
(86) PCT No.: PCT/DE2019/100068

§ 371 Date: Jul. 23, 2020

(30) Foreign Application Priority Data
Jan. 23, 2018 (DE) 20 2018 100 353.4

(51) Int. Cl. A61B 17/70; A61B 17/86

(52) U.S. Cl. CPC A61B 17/7032; A61B 17/863

(57) ABSTRACT

The invention relates to a surgical screw for fixing in a bone of a human or animal body, having a head, having a shaft, and having an external thread. The external thread is disposed on at least one segment of the shaft of the surgical screw. The external thread extends onto at least one segment of the head.

### CROSS-REFERENCE TO RELATED APPLICATIONS

This application is a national phase application of and claims priority to PCT application PCT/DE2019/100068 filed on Jan. 23, 2019, which claims priority to German Patent Application No. 20 2018 100 353.4, filed Jan. 23, 2018, and titled INTRAVERTEBRAL SCREW, all of which are incorporated by reference in its entirety herein.

### FIELD OF INVENTION

The invention relates to a surgical screw for inserting into the bone of a human or animal body, according to the preamble of claim 1.

### BACKGROUND

A pedicle screw comprising a shaft having a thread disposed on the exterior thereof is known from CH 703 411 A2. A head is provided at the rear end of the screw. A tool is applied to the head in order to cause the pedicle screw to rotate about the longitudinal axis thereof. The pedicle screw is screwed into the bone of a human and/or animal body by the rotation.

Known surgical screws for inserting into a bone are disadvantageous because the head of the screw abuts the shaft of the screw. The large diameter of the head is thereby adjacent to the smaller, relative to the head, diameter of the shaft of the surgical screw. The head of the surgical screw can break off of the shaft as the load increases.

For other known surgical screws, the entire head protrudes out of the bone after screwing into the human bone. There is thereby a risk that the head of the screw will scratch or injure adjacent organs in the body.

The head of the screw protruding from the one protrudes from the bone after a successfully completed operation in a manner palpable to the patient.

This is particularly disadvantageous if the screw is screwed into the bone from the front or the side with respect to the human body.

If the head of the screw protrudes entirely from the bonne, then the center of gravity of the screw is relatively far out at the outer periphery of the human body. As the load on the screw increases, this can cause damage to the bone.

### SUMMARY

The object of the invention is achieved by the features of claim 1.

The invention relates to a surgical screw for inserting into the bone of a human or animal body.

The invention relates to a surgical screw, preferably a modified intravertebral screw. The surgical screw can also be a pin.

It is assumed below that the surgical screw is an intravertebral screw.

### Surgical Screw in the Form of an Intravertebral Screw

The intravertebral screw refers to a surgical instrument for stabilizing the spinal column in the form of a screw. However, the surgical screw, particularly the intravertebral screw, can of course be inserted into any other bone of the human or animal skeleton.

The intravertebral screw can also be implemented as a corticalis screw. The corticalis screw serves for surgical treatment of fractures. The corticalis screw comprises an external thread having profile roots and peaks in the bone.

Tightening the corticalis screws brings about a continuous pressure during the operation. Said pressure causes a pretension to be reached, providing additional stability to the bone structure.

Greater corticalis anchoring is achieved by means of the invention.

The intravertebral screw comprises a shaft. The intravertebral screw comprises a conical tip at one end of the shaft for more easily inserting the intravertebral screw into the bone.

A head is provided at the opposite end of the intravertebral screw.

The head of the intravertebral screw comprises shaped elements for bringing into the contact with a tool for performing a radial motion by the intravertebral screw.

The screw comprises an external thread at least in regions.

The external thread is disposed radially on the outer circumference of the shaft at at least one segment of the shaft of the intravertebral screw.

The external thread of the intravertebral screw is disposed radially on the outer circumference of the head at at least one segment of the head.

The intravertebral screw can be permanently anchored in the bone. Alternatively, the intravertebral screw is removed from the bone after a predetermined period of time.

The intravertebral screw can be connected to a surgical stabilizing element at the free end thereof opposite the tip thereof.

The surgical stabilizing element can be disposed outside of the body or in the interior of the human body.

The intravertebral screw preferably, but not exclusively, comprises a cylindrical shape.

The intravertebral screw comprises an external thread engaging in the bone.

The interlocked screw is anchored in the bone in a force-fit and/or form-fit manner. The anchoring is preferably done by means of an external thread. Any other form of anchoring in the bone can be selected, however. The connection can be released.

The intravertebral screw is made of a non-rusting steel. The intravertebral screw can also be made of a metal alloy or of ceramic or of plastic.

Any material tolerated by the body and particularly not bringing about any allergic and/or rejection reaction can be used for producing the intravertebral screw. For example and non-exclusively, the intravertebral screw is made of a bone. Said screw can also be made of carbon or any other material.

The intravertebral screw is anchored in the bone. Alternatively, the intravertebral screw can penetrate through the bone. A surgical stabilizing element can be disposed at both sides of the bone.

The intravertebral screw particularly absorbs transvers loads.

The intravertebral screw comprises a uniform diameter over the entire screw length. Alternatively, the screw can have different shaft diameters along the length of the shaft.

The arrangement of the intravertebral screw is found to be advantageous because the screw can have a different length and/or a different diameter, depending on the characteristics of the bone.

### Head and Neck of the Intravertebral Screw

The head of the intravertebral screw comprises shaped elements.

The shaped elements serve for engaging with tools. The engaging of each tool into the shaped element of the head serves for causing the intravertebral screw to rotate about the longitudinal axis thereof.

The intravertebral screw is screwed into the bone by the rotation about the longitudinal axis thereof.

The head of the intravertebral screw can be made of the same material as the shaft of the intravertebral screw. Of course, the head of the intravertebral screw can also be made of a different material.

The head of the intravertebral screw can be implemented as a flat head, a seating head, a round head, a hex head, or a countersunk head, an oval head, a cylinder head, a round flathead, a pan head, or a milled countersunk head.

The tool can also be a screwdriver.

For disposing the tool in a force-fit and/or form-fit arrangement of the tool at the head of the intravertebral screw, the shaped elements can be disposed at the outer circumference of the head of the interlocked screw.

The shaped elements of the head can also be disposed at the end of the head facing away from the tip of the intravertebral screw.

The head of the intravertebral screw can comprise a square socket drive, a hexagonal socket drive, or a polygonal socket drive.

Any other screw head drive can also be selected for inserting the intravertebral screw into the bone.

According to the invention, the head of the intravertebral screw comprises an external thread at at least one segment of the head.

It is thereby achieved that both the shaft and the segment of the head comprising the external thread of the intravertebral screw penetrate into the bone when inserting into the bone.

The head of the intravertebral screw comprises a hole serving for receiving orthopedic stabilizing elements preferably running perpendicular to the shaft of the intravertebral screw.

In this manner, at least two intravertebral screws can be connected to each other, preferably disposed parallel to each other.

The orthopedic stabilizing element for connecting at least two intravertebral screws can be implemented in the form of a bar.

The stabilizing element connects at least two bones of the skeleton to each other. Said element can be used for connecting at least two regions of a single bone.

The stabilizing element can be a bar or a plate. Alternatively, the stabilizing element can be a cable. Of course, the orthopedic stabilizing element can also be a different object or can have a different geometric shape.

The stabilizing element can be an elastic or a non-elastic body. The stabilizing element can remain in the body after the stabilizing has taken place. Alternatively, the stabilizing element is removed from the body again after the operation.

Hexagonal screws are preferably provided in and/or on the head for fixing the orthopedic stabilizing element. Alternatively, a nut embedded in the head can be provided for attaching the orthopedic stabilizing element. The orthopedic stabilizing element can be inserted laterally in the hole of the head. A perpendicularly running screw is provided for fixing the orthopedic stabilizing element on the head.

Alternatively, different means for fixing the orthopedic stabilizing element can also be provided in the head.

A neck can be disposed between the shaft of the intravertebral screw and the head of the intravertebral screw.

The external thread is disposed radially on the neck at least at one segment of the neck.

The external thread disposed on the shaft transitions into the external thread disposed on the neck.

The external thread of the intravertebral screw disposed on the neck transitions into the external thread of the head of the intravertebral screw.

The neck can have the same diameter as the shaft of the intravertebral screw.

The neck can have a diameter corresponding to the diameter of the head of the intravertebral screw. Alternatively, the neck of the intravertebral screw can have a diameter increasing in the direction of the head for connecting the shaft to the head.

The shaft and the neck and the head of the neck have different diameters from each other.

Alternatively, the shaft and the neck and the head of the surgical screw, particularly the intravertebral screw, can match with respect to the diameter thereof.

The diameter can vary over the length of the head of the intravertebral screw and/or over the length of the neck of the intravertebral screw.

Such a design has the advantage that the intravertebral screw is particularly well anchored in the bone.

It is provided that the head of the intravertebral screw is connected to the shaft by a hinge.

The head can also be mounted on the shaft of the intravertebral screw in a different technical manner. The invention has been found to be advantageous because the intravertebral screw can be preferably screwed into a vertebra of the spinal column from the front, from the side, or from the rear.

An external thread is disposed on at least one segment of the head of the intravertebral screw.

According to the design, the segment of the head of the intravertebral screw having the external thread also penetrates into the bone in addition to the shaft of the intravertebral screw.

In addition, the neck disposed between the shaft and the head penetrates into the bone.

When the intravertebral screw remains in the bone of a patient, an intravertebral screw having a relatively shorter shaft can thus be selected.

Another advantage is that the head of the intravertebral screw protrudes only to a slight extent out of the bone.

Only the segment of the head of the intravertebral screw not having an external thread protrudes out of the bone.

The shaft of the intravertebral screw can be shorter in the axial extent, in that the head of the intravertebral screw has an external thread.

The head of the intravertebral screw is inserted into the bone to the extent that an external thread is disposed on the head in addition to the shaft of the intravertebral screw.

It has been found to be advantageous that the intravertebral screw can be screwed into hollow bones, into flat bones, into short bones, into sesamoid bones, and into air-filled and irregular bones.

Due to the neck, the external thread of the shaft of the intravertebral screw can be transitioned and/or extended to the external thread of the head of the intravertebral screw.

The screw head and/or the neck of the intravertebral screw becomes a functional component of the external thread. According to the invention, improved anchoring of the intravertebral screw in the bone is achieved.

According to the design, the intravertebral screw can have a funnel shape from the tip of the intravertebral screw to the head thereof.

The funnel shape enables particularly solid and uniform anchoring of the intravertebral screw in the bone.

A further advantage of the invention is that shafts, necks, and heads of different designs and embodiments can be combined with each other when produced.

Due to the external thread of the head penetrating into the bone, the profile of the intravertebral screw protruding out of the bone is reduced.

### External Thread

The external thread is disposed at least one a segment of the shaft of the intravertebral screw and at least on a segment of the head. An external thread is preferably disposed on the neck of the intravertebral screw between the shaft and the head thereof.

The term external thread is understood to mean a profiled notching in the shape of a spiral, continuously or at least in segment extending about the outer circumference of the shaft and about the outer circumference of the neck and about the outer circumference of the head of the intravertebral screw. The profiled notching comprises a profile peak. The profile peak runs between two profile roots.

The external thread is implemented as a right-hand thread. Alternatively, the external thread can be implemented as a left-hand thread in each case.

The external thread on the shaft and/or on the neck and/or on the head of the intravertebral screw is implemented as a self-tapping thread in each case. Of course, the thread turns on the shaft, on the neck, and on the head of the intravertebral screw can also have a different thread shape.

According to the invention, the neck of the intravertebral screw comprises an external thread. Alternatively, the neck of the intravertebral screw can also be disposed between two thread turns.

The neck of the intravertebral screw can have the same diameter as the shaft of the intravertebral screw.

Alternatively, the neck of the intravertebral screw can have a diameter increasing in the direction of the head.

Purely as an example, and in no way exclusively, the diameter of the shaft in relation to the cumulative axial length of the shaft and of the segment of the head having an external thread can be 4.5 mm×20 mm or 6.5 mm×20 mm or 7.5 mm×20 mm.

Also purely as an example, and in no way exclusively, the diameter of the external thread on the head of the intravertebral screw can be 12.5 mm.

In a further example, the axial length of the segment of the head of the intravertebral screw having an external thread is 6.35 mm.

The remaining segment of the head having no external thread can have a length of 12 mm, purely as an example.

In a further example, the spacing between two thread turns in the region of the neck of the intravertebral screw is 4.6 mm.

In a different embodiment example, the spacing between two thread turns in the region of the neck of the intravertebral screw can be 10.2 mm.

All of the above diameter and length information is purely intended as an example and can also have different values.

### Channel

A channel is disposed, extending axially along the longitudinal axis of the intravertebral screw through the shaft of the intravertebral screw. The channel runs through the neck and/or through the head of the intravertebral screw. The channel extends along the longitudinal axis of the intravertebral screw from the head of the intravertebral screw to the tip thereof.

The channel is implemented for at least one object.

A wire or an internal screw can be inserted in the channel along the longitudinal axis of the intravertebral screw. The internal screw is a screw for inserting in the intravertebral screw and having a smaller diameter relative to the intravertebral screw. Of course, a different object, such as a cannula, can also be inserted in the intravertebral screw.

The object for inserting in the channel in the interior of the shaft and/or of the head and/or of the neck of the intravertebral screw is an internal screw or a bolt or a pin.

The object is screwed into the channel. The object can be pushed or pulled into the channel.

Alternatively, the object can be pulled into the channel of the intravertebral screw. The pulling device can be a thread or a chain, for example, and in no way exclusively.

The object can have a smaller diameter than the channel.

The channel serves as a target device or line for precisely locating the intravertebral screw in the bone.

A spreading device is further disposed. The object inserted into the intravertebral screw thereby has a greater diameter than the channel in the interior of the intravertebral screw.

For example, and not exclusively, the channel has a diameter from 2 to 3 mm. The diameter can also take on a different value.

By inserting the object having the greater diameter into the channel, the shaft and/or the neck and/or the head of the intravertebral screw is spread apart.

By inserting the object having the greater diameter into the channel, the shaft and/or the neck and/or the head of the intravertebral screw can be deformed.

Alternatively, the shaft and/or the neck and/or the head of the intravertebral screw can retain the previously shape thereof, so that only the external thread of the shaft and/or of the neck and/or of the head is deformed.

The object inserted in the intravertebral screw is pulled out of the intravertebral screw again at least in regions. The tip of the intravertebral screw is thereby drawn along, at least in pieces. The segment of the shaft becomes thicker and rounder relative to the surrounding bone in this manner.

The shaft thereby gains additional surface area. The head forms a counter-anchor to the head of the intravertebral screw relative to the surrounding bone.

After expanding, the intravertebral screw spreads radially in a manner similar to an anchor into the bone.

By expanding the intravertebral screw (3), the anchoring of the screw in the bone can be additionally improved.

### Characteristic Values

The characteristic values of the external thread are the flank diameter or the core diameter or the pitch angle of the thread.

The invention also refers to the multi-start or single-start nature of the external thread as a core characteristic value of the external thread.

The flank diameter thereby refers to the diameter of an imaginary geometric circular cylinder intersecting the thread profile in such a manner that the width of the profile roots and the profile peaks thus arising are identical in size.

The core diameter of the external thread refers to the least diameter of the thread geometry.

The term pitch angle means the angle between the flank of the external thread and a longitudinal axis of the intravertebral screw.

According to the invention, a further characteristic value of the external thread is the multi-start or single-start nature of the external thread.

The single-start external thread runs in the form of a profiled notching in a spiral manner about the surface of the intravertebral screw.

For a multi-start external thread, a plurality of thread turns run parallel to each other.

The characteristic values of the external thread disposed on the shaft and of the external thread disposed on the neck and of the external thread disposed on the head are identical.

Alternatively, the characteristic values of the external thread disposed on the shaft and of the external thread disposed on the neck and of the external thread disposed on the head can be different from each other.

### DESCRIPTION OF THE DRAWINGS

Further advantages and embodiments of the invention can be seen in the following drawing. Shown are:

FIG. 1 shows two vertebrae of a human skeleton having intravertebral screws,

FIG. 2 shows part of an intravertebral screw having a shaft, a neck, and a head, and

FIG. 3 shows an intravertebral screw having a neck comprising an external thread.

### DETAILED DESCRIPTION

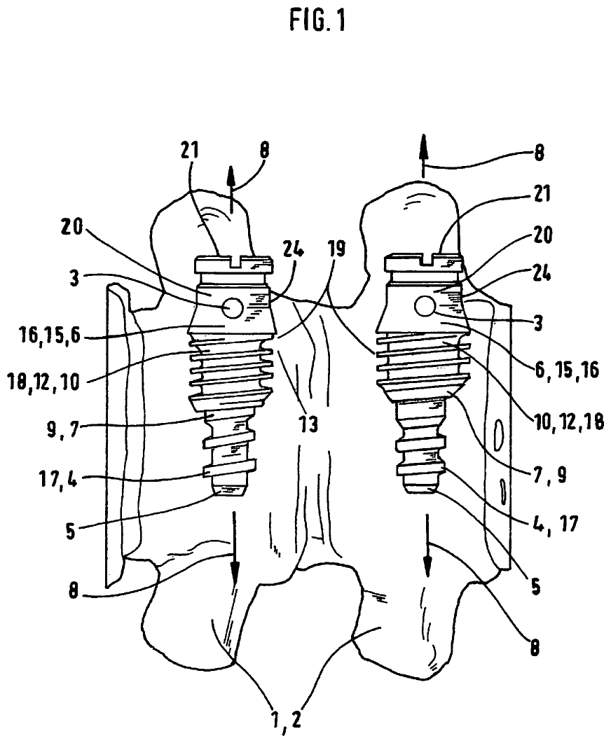

FIG. 1 shows two adjacent bones 1 in the form of vertebrae 2 of a human skeleton.

One intravertebral screw 3 is screwed into each of the vertebrae 2 from the side. The intravertebral screws 3 are disposed approximately perpendicular to the spinal column of the skeleton.

In FIG. 1, the intravertebral screw 3 comprises a shaft 4. The intravertebral screw 3 comprises a tip 5 for penetrating the shaft 4 into the vertebra 2.

A head 6 is disposed at the end of the intravertebral screw 3 facing away from the tip 5.

The intravertebral screw 3 has a neck 10 between the shaft 4 and the head 6.

An external thread 7 is disposed on the shaft 4 along a longitudinal axis 8 of the intravertebral screw 3. The external thread 7 extends in a segment 9 of the shaft 4.

One external thread 7 is disposed on each segment 12 of the neck 10.

One segment 13 of the head 6 comprises an external thread 16 on the outer circumference 15 of the head 6.

The external thread 17 transitions into the external thread 18 on the neck 10.

According to the depiction in FIG. 1, the external thread 18 of the neck transitions into the external thread 16 of the head 6.

The external thread 17 of the shaft 4 and the external thread 18 of the neck 10 and the external thread 16 of the head 6 each have different diameters.

A segment 19 of the head 6 is anchored in the vertebra 2 in the region of the external thread 16 thereof.

A segment 20 of the head 6 does not have an external thread 16.

The segment 20 of the head 6 protrudes from the vertebra 2.

The head 6 comprises a shaped element 21 at the end of the head 6 facing away from the tip 5.

A tool (not shown) engages in the shaped element 21 of the head 6.

The intravertebral screw 3 is caused to rotate about the longitudinal axis 8 thereof by means of the tool.

The intravertebral screw 3 is screwed into the bone 1, the vertebra 2 in the case of FIG. 1, by the rotation.

The heads 6 of the intravertebral screws 3 each comprise a hole 24 approximately parallel to the axis of the spinal column.

The holes 24 shown in FIG. 1 are aligned with each other.

A bar (not shown) is inserted through the holes 24 of the heads 6 of the intravertebral screws 3 for connecting the two intravertebral screws 3 shown in FIG. 1.

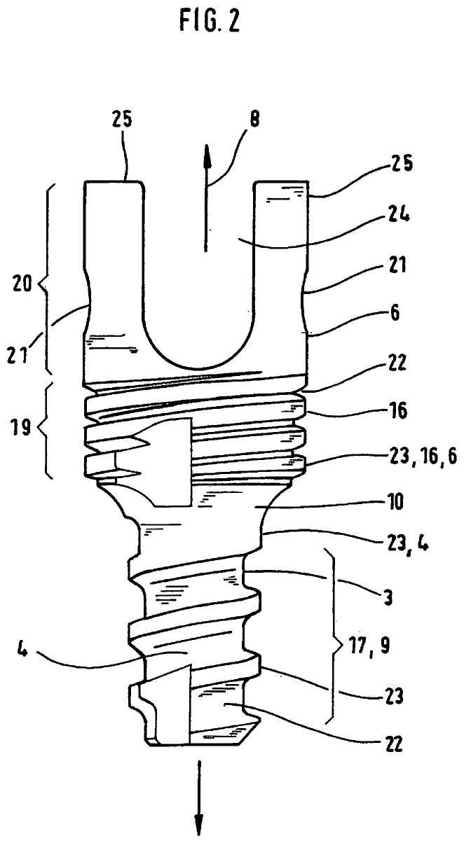

FIG. 2 shows an intravertebral screw 3. The intravertebral screw 3 comprises the shaft 4 and the head 6.

A neck 10 between the shaft 4 and the head 6 is shown in FIG. 2.

The head 6 comprises a hole 24 open to the top and approximately perpendicular to the longitudinal axis 8 of the intravertebral screw 3.

A bar (not shown) is inserted into the hole 24 approximately perpendicular to the longitudinal axis 8 of the intravertebral screw 3.

The head 6 comprises a U-shaped recess formed by at least two exterior walls 25 for forming the hole 24. The recess serves for receiving the bar.

The external thread 17 is disposed on the shaft 4 in the segment 9.

The external thread 17 of the shaft 4 comprises profile roots 22 and profile peaks 23.

The neck 10 of the intravertebral screw 3 is disposed between two profile peaks 23 of the external thread 17 of the shaft 4 and of the external thread 16 of the head 6 in FIG. 2.

The external thread 16 of the segment 19 of the head 6 protrudes into the bone 1 (not shown).

The segment 20 of the head 6 of the intravertebral screw 3 protrudes out of the bone 1 (not shown) after screwing the intravertebral screw 3 into said bone.

The profile roots 22 and the profile peaks 23 in the external thread 16 of the head 6 are shown in FIG. 2.

Shaped elements 21 are shown on the head 6. The shaped elements 21 serve for engaging tools by means of which the intravertebral screw 3 is screwed into the bone (not shown).

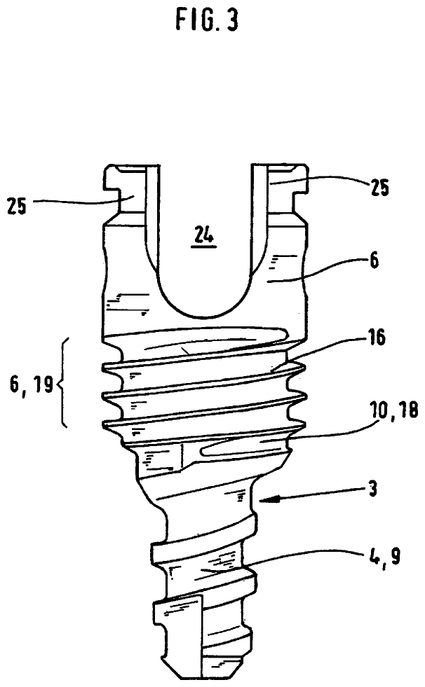

FIG. 3 shows an intravertebral screw 3 according to FIG. 2 having the difference that the neck 10 of the intravertebral screw 3 comprises an external thread 18 between the shaft 4 and the head 6.

After screwing the intravertebral screw 3 into the bone (not shown), the shaft 4 and the neck 10 of the intravertebral screw 3 protrude into the bone.

The segment 19 having the external thread 16 of the head 6 of the intravertebral screw 3 shown in FIG. 3 protrudes further into the bone (not shown) after screwing into the bone.

The head 6 comprises a U-shaped recess formed by at least two exterior walls 25 for forming the hole 24 in FIG. 3. The recess serves for receiving the bar.

### REFERENCE NUMERALS

1 Bone
2 Vertebra
3 Intravertebral screw
4 Shaft
5 Tip
6 Head
7 External thread
8 Longitudinal axis
9 Shaft segment
10 Neck
11 Subject to change
12 Neck segment
13 Head segment
14 Subject to change
15 Head circumference
16 Head external thread
17 Shaft external thread
18 Neck external thread
19 Segment of the head in the bone
20 Segment of the head protruding from the bone
21 Shaped element
22 Profile root
23 Profile peak
24 Hole in head
25 Outer wall

1. A surgical screw for fixing in a bone of a human or animal body, comprising a head, having a shaft, and having a first external thread disposed on at least one segment of the shaft of the surgical screw, further comprising a second external thread that extends onto at least one segment of the head.

2. The surgical screw according to claim 1, wherein the surgical screw is an intravertebral screw.

3. The surgical screw according to claim 1, wherein the head comprises a U-shaped recess formed by at least two outer walls for receiving a stabilizing element.

4. The surgical screw according to claim 1, wherein the surgical screw comprises a neck between the shaft and the head, wherein a third external thread extends onto the neck.

5. The surgical screw according to claim 1, wherein the first external thread transitions into the third external thread disposed on a neck, and/or the third external thread disposed on the neck transitions into the second external thread disposed on a circumference of the head.

6. The surgical screw according to claim 4, wherein characteristic values of the first external thread and the third external thread and the second external thread are identical.

7. The surgical screw according to claim 6, wherein the characteristic values of the first external thread disposed on the shaft and the third external thread disposed on the neck and the second external thread disposed on the head are different from each other.

8. The surgical screw according to claim 6, wherein the characteristic values of the first, second, and third external threads are a flank diameter or a core diameter or a pitch angle of the first, second, and third external threads or a multi-start or single-start nature of the first, second, and third external threads.

9. The surgical screw according to claim 1, wherein the head is joined to the shaft of the surgical screw by means of a hinge.

10. The surgical screw according to claim 3, wherein the head comprises a hole running horizontal to the shaft and implemented for passing through the stabilizing element.

11. The surgical screw according to claim 10, wherein the stabilizing element is a bar and/or a plate or a cable connecting at least two bones or at least two regions of the bone to each other.

12. The surgical screw according to claim 5, wherein the shaft and the neck and/or the neck and the head of the surgical screw each transition conically into each other.

13. The surgical screw according to claim 4, wherein the shaft and the neck and the head of the surgical screw each have a diameter deviating from each other.

14. The surgical screw according to claim 4, wherein the shaft and the neck and the head of the surgical screw each have an identical diameter.

15. The surgical screw according to claim 4, further comprising an axial channel extending along a longitudinal axis through the shaft and/or through the neck and/or through the head.

16. The surgical screw according to claim 15, wherein a tension device is disposed for introducing at least one object into the channel.

17. The surgical screw according to claim 15, wherein a wire or an internal screw can be introduced into the channel.

18. The surgical screw according to claim 15, wherein a spreading device is disposed for expanding the shaft and/or the neck and/or the head.
